(19) United States
(12) Patent Application Publication
(10) Pub. No.: US 2021/0260371 A1
(43) Pub. Date: Aug. 26, 2021

(54) PATIENT THERAPY SYSTEMS AND METHODS

(71) Applicant: CyMedica Orthopedics, Inc., Scottsdale, AZ (US)

(72) Inventors: Kereshmeh Shahriari, Cave Creek, AZ (US); Dharmavirsinh Bharatsinh Jhala, Scottsdale, AZ (US)

(21) Appl. No.: 17/205,571

(22) Filed: Mar. 18, 2021

Related U.S. Application Data
(63) Continuation of application No. 16/208,329, filed on Dec. 3, 2018.
(60) Provisional application No. 62/594,336, filed on Dec. 4, 2017.

(51) Int. Cl. A61N 1/36; A61N 1/04; A61F 5/01

(52) U.S. Cl. CPC A61N 1/36003; A61N 1/3603; A61N 1/0484; A61N 1/0452; A61F 2002/701; A61F 5/0125; A61N 1/36157; A61N 1/0492; A61N 1/36153; A61N 1/36021

(57) ABSTRACT

Some embodiments include a knee therapy system that includes a flexible garment or wrap that is electrically conductive, and a range-of-motion sensor coupled or integrated with the flexible garment or wrap. The sensor is coupled or integrated with the flexible garment or wrap, and includes a plurality of electrodes including an active electrode and a receiving electrode. The electrodes can be in physical contact with skin of a patient forming an electrical circuit with control electronics of the controller. The electrical circuit measures an electrical parameter using the active and receiving electrodes, and forms a closed loop electrical muscle stimulation system, where stimulation current or voltage is applied by the electrodes onto the skin between the active and receiving electrodes based on a program and an electrical parameter measured through the electrodes. An optical sensor or camera can be configured to track body joints of a user.

### RELATED APPLICATIONS

This application is a Continuation Application of U.S. patent application Ser. No. 16/208,329, filed Dec. 2, 2018, which claims priority to U.S. Provisional Application No. 62/594,336, filed Dec. 4, 2017, the entire contents of which are incorporated herein by reference.

### BACKGROUND

Orthopedic braces and wraps are useful as preventative aids to prevent injuries to joints caused by motions or orientations of the joint that are outside the biomechanical limits of the joint. Orthopedic braces and wraps are also useful to promote proper healing of a joint following an injury to, or surgery on, the joint, and can be used to stabilize joints with arthritis, thereby alleviating pain.

Knee osteoarthritis (OA) is quite common, and is diagnosed and defined as a loss of hyaline cartilage within the joint. However, muscle weakness and impairments associated with the disease may be the primary underlying cause of functional impairments, and muscle weakness and/or dysfunction may actually precede and expedite the cartilage deterioration. Some studies have suggested quadriceps weakness plays a significant role in the OA disease progression. Often, the strengthening components of quadriceps rehabilitation are focused on exercises that require patients to voluntarily activate and contract their own muscles. However, if the patient fails to overcome muscle inhibition, they are unable to achieve goals to retard atrophy and regain full strength. Recent clinical studies have shown that the addition of an at home LAMES therapy system can help manage and improve knee OA symptoms by removing the barrier of muscle inhibition on behalf of the patient, and activating the quadriceps muscle.

If the patient has been fitted with a brace, a physical therapist may manually adjust the brace based on guidelines provided by a physician to reduce or increase the allowed motion of the injured joint, or to adjust a brace that has become loose secondary to muscle atrophy, or both. These manual adjustments often lead to errors, as the adjustments are based on the personal judgments of the physical therapist (or medical professional), and the muscles and surrounding tissues may not be of sufficient strength to support the joint.

In some cases, the patient may receive electrical muscle stimulation (EMS) at the start of the physical therapy process to regain the ability to voluntarily contract their muscles before exercising and stretching begins. EMS, also known as neuromuscular electrical stimulation (“NMES”), has been used in therapeutic practice virtually unchanged in the last 30 years. The current use model involves taking a target muscle group and providing electrical stimulation to mimic the action potentials normally created from neurological signals to activate and elicit an action potential and resultant contraction of the muscle fibers causing the muscle to contract. The electrical stimulation therapy can be enhanced by determining the appropriate level of power and/or duration of the electrical pulse, the pulse width, the phase characteristics (monophasic, biphasic, triphasic, polyphasic, symmetric), frequency, waveform shapes (sinusoidal, square, triangular, trapezoidal, sawtooth, custom), duty cycle, work cycle on/off times, work cycle ramp type. EMS is also used by the therapist (as prescribed by the health care provider) to strengthen muscles which have atrophied.

Some other clinical conditions in need of improved therapies include stress incontinence, urge incontinence, urinary incontinence, and urinary leakage caused by a weak pelvic floor muscle. The lower pelvic muscles may become damaged or weakened through childbirth, lack of use, aging, or as the collateral result of surgical procedures (e.g. prostatectomy). Current therapies include intra-vaginal or intra-rectal electrical stimulation using insertable probes, sacral nerve stimulation using surgical implantable stimulators, physical therapy, Kegel exercises, medications for bladder control, etc.

However, the delivery of EMS for muscle strengthening is sub-optimal, as it is usually performed when the patient is with the therapist. Further, a physician (e.g., a surgeon) treating a patient often sees the patient several times after the treatment of the injury or condition (e.g., surgery). The physician typically determines the next step in the patient's treatment based on an overall assessment of the patient's condition during a visit. However, the physician typically does not have comprehensive and objective data associated with the patient's injury that could be used to help in the physician's assessment of the patient and the next step in the patient's treatment. Specifically, the physician may not be able to obtain accurate ranges of joint motion or muscle strength since the last visit. Consequently, the physician often determines the patient's next course of treatment based on his or her subjective analysis of the patient at the time of the patient's visit; this analysis may be sub-optimal. In addition to the data being sub-optimal, the time points at which these data are observed is inefficient and sub-optimal. The patient may heal faster or slower than a typical patient, and the patient's treatment may be able to be better customized to his/her actual progress.

As the health care system in the United States transitions toward value-based care such as implementation of the Medicare Merit-based Incentive Program (MIPS), there is an increased emphasis on paying the providers for quality and engaging patients with their care for different type of diseases. The addition of a home-based muscle strengthening program that allows for patient's strengthening of quadriceps, reduction of pain, patient reported outcomes, remote monitoring of the patient's progress, and engagement in their care path could significantly decrease healthcare costs through a reduction in additional costly therapeutic options including total knee arthroplasty procedures.

### SUMMARY

Some embodiments include a knee therapy system comprising a flexible garment or wrap, where at least a portion of the flexible garment or wrap is electrically conductive, and a plurality of electrodes coupled to or integrated with the flexible garment or wrap. Some embodiments include at least one range-of-motion sensor coupled to or integrated with the flexible garment or wrap. In some embodiments, a plurality of electrodes includes at least one active electrode and at least one receiving electrode. In some embodiments, the electrodes are configured and arranged to be coupled with skin of a patient forming an electrical circuit with control electronics of at least one controller. In some embodiments, the electrical circuit is configured and arranged to measure an electrical parameter using the at least one active electrode and at least one receiving electrode, and to form a closed loop electrical muscle stimulation system. Further, a stimulation current or voltage applied onto the skin between the at least one active electrode and at least one receiving electrode is based on at least one program and at least one electrical parameter measured through the at least one active electrode and at least one receiving electrode. Further, the at least one controller is configured and arranged to (a) apply a sense electrical pulse to the tissue, (b) measure the at least one electrical parameter from the tissue, (c) using at least one of the active electrodes, adjustably apply a stimulation pulse to the tissue based at least in part on the measured electrical parameter. Further, the stimulation is adjustably controlled by the at least one controller to maintain a constant power output to the tissue based at least in part on the at least one electrical parameter, and (d) repeat steps (a)-(c).

In some embodiments, the flexible garment or wrap comprises a popliteal cutout. Some embodiments further comprise a brace assembly integrated or coupled to the flexible garment or wrap. In some embodiments, the brace assembly includes at least one brace element or stay coupled with the flexible garment or wrap. In some embodiments, the brace assembly includes a dial hinge. In some embodiments of the invention, the dial hinge includes range-of-motion (ROM) stops configured to enable a wearer to achieve a customized fitting and therapy.

Some embodiments of the invention include a controller configured to be coupled to at least one computer readable medium that is configured to store usage data relating to the patient's use of the therapy system. In some embodiments, the at least one controller is coupled to an outer surface of the flexible garment or wrap. Some embodiments further comprise at least one range-of-motion sensor coupled to or integrated with the flexible garment or wrap.

Some embodiments include at least one wireless transmitter coupled to the controller. In some embodiments, the flexible garment or wrap comprises a compressible and non-slip material. In some embodiments, the flexible garment or wrap is configured to be secured to a wearer by at least one hook-and-loop fastener.

Some embodiments further comprise a computing program, applet or application configured to transfer usage data using the at least one wireless transmitter. In some embodiments, the at least one controller is configured and arranged to electromagnetically couple with a mobile computing device using at least a portion of the computing program, applet or application.

In some embodiments, at least a portion of the computing program, applet or application is configured and arranged to display a user interface on a user's computing device, and the user interface configured to display at least some usage data and to enable control of a parameter through the at least one controller.

In some embodiments, the usage data includes a user's compliance to certain daily movements and/or one or more physiotherapy or exercise routines. In some embodiments, the usage data comprises kinematic data, including orientation data and acceleration data.

In some embodiments of the invention, the at least one sensor comprises an accelerometer, and/or a motion sensor, a proximity sensor, and/or an optical sensor, and/or a motion sensor, and/or a gyrometer, and/or a magnetometer, and/or a proximity sensor, and/or a hydration sensor, and/or a force or pressure sensor, and/or a position sensor, and/or a global positioning sensor (GPS), and/or an optical sensor, and/or a magnetic sensor, and/or a magnetometer, and/or an inductive sensor, and/or a capacitive sensor, and/or an eddy current sensor, and/or resistive sensors, and/or a magneto-resistive sensor, and/or an inductive sensor, and/or an infrared sensor, and/or an inclinometer sensor, and/or a piezoelectric materials or piezoelectric-based sensor, and/or a blood-oxygen sensor, and/or a heart-rate sensor, and/or a laser or ultrasound based sensor, and/or an electromyography type sensor. Some embodiments include at least one sensor or range-of-motion sensor comprising an optical sensor or camera configured to track body joints of a user.

Some embodiments include an assembly comprising an electrically conductive flexible garment or wrap, and at least one range-of-motion sensor coupled to or integrated with the flexible garment or wrap, at least one controller, and at least one wireless transmitter coupled to the controller. Some embodiments include a plurality of electrodes coupled to or integrated with the flexible garment or wrap. In some embodiments, a plurality of electrodes including at least one active electrode and at least one receiving electrode is coupled to or integrated with the flexible garment or wrap, and configured and arranged to be in physical contact with skin of a patient forming an electrical circuit with control electronics of the at least one controller. In some embodiments, the electrical circuit is configured and arranged to measure an electrical parameter using the at least one active electrode and at least one receiving electrode, and to form a closed loop electrical muscle stimulation system. Further, the stimulation current or voltage applied onto the skin between the at least one active electrode and at least one receiving electrode is based on at least one program and at least one electrical parameter measured through the at least one active electrode and at least one receiving electrode. Further, the at least one controller is configured and arranged to (a) apply a sense electrical pulse to the tissue, (b) measure the at least one electrical parameter from the tissue, (c) using at least one of the active electrodes, adjustably apply a stimulation pulse to the tissue based at least in part on the measured electrical parameter. Further, the stimulation is adjustably controlled by the at least one controller to maintain a constant power output to the tissue based at least in part on the at least one electrical parameter, and (d) repeat steps (a)-(c). In other embodiments of the assembly, the controller is configured to transmit usage data to at least one computer readable medium, the usage data relating to the patient's use of the assembly.

### DESCRIPTION OF THE DRAWINGS

FIG. 1 illustrates a knee therapy system in accordance with some embodiments of the invention.

FIGS. 2 and 3 illustrate partial inner side views of a knee wrap of the knee therapy system of FIG. 1 in accordance with some embodiments of the invention.

FIG. 4 illustrates an inner view of the knee wrap in accordance with some embodiments of the invention.

FIG. 5A illustrates a front side perspective view of a brace system comprising a combined modular orthopedic brace and conductive wrap in accordance with some embodiments of the invention.

FIG. 5B illustrates a front view of a brace system comprising a combined modular orthopedic brace and conductive wrap in accordance with some embodiments of the invention.

FIG. 6 illustrates a combined modular orthopedic brace and conductive wrap in an open view position in accordance with some embodiments of the invention.

FIG. 7 illustrates an inner region of a brace showing two contact points used to determine if the brace is being worn by a human in accordance with some embodiments of the invention.

FIG. 8 illustrates an inner region of a brace system with sensors and electrodes in accordance with some embodiments of the invention.

FIG. 9 depicts a knee brace system wireless data transfer data architecture in accordance with some embodiments of the invention.

FIG. 10 depicts wireless data transfer data architecture between a knee brace and a controller in accordance with some embodiments of the invention.

FIG. 11 illustrates a computer system controller in accordance with some embodiments of the invention.

FIG. 12 illustrates a computer system including a backend server in accordance with some embodiments of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

Some embodiments include assemblies, components, systems and methods for providing EMS therapy for muscle strengthening. Some embodiments include a systems and methods that measure the outcomes of the therapy in real-time using one or more combinations of the assemblies, components, systems, and methods of use. In some embodiments, any of the apparatus, assemblies, components, and/or systems described herein can be configured to provide transcutaneous electrical nerve stimulation (“TENS”) therapy. In some embodiments, any of the apparatus, assemblies, components described herein can be configured to provide neuromuscular electrical stimulation (“LAMES”) therapy. In some further embodiments, any of the apparatus, assemblies, components described herein can alternately, selectively, and/or substantially simultaneously provide LAMES and/or TENS therapy.

In some embodiments of the invention, any of the brace therapy systems and methods described herein can include a device that provides EMS therapy and measurements of the associated outcomes simultaneously. Most of the brace therapy systems and methods disclosed focus on providing therapy for knee osteoarthritis (“OA”), however, some or all the assemblies, components, and methods can be applied to other therapeutic applications including OA in all joints.

In some embodiments, the outcomes of the therapy can be assessed by measuring joint range of motion, and or joint mobility, and/or joint angles, and/or joint loads. In some embodiments, the joint range of motion, and or joint mobility, and/or joint angles, and/or joint loads can be measured using one or more inertial measurement units (“IMU”).

In some embodiments of the invention, the various electronic components can be integrated into one or more modules of a knee brace system, and the modules can be combined and recombined into various configurations. In some embodiments, some knee brace systems or assemblies can comprise a set of modules each of which has a distinct function, and the combination of which creates a general LAMES platform with different user interfaces and/or different sensors for data collection. In some embodiments of the invention, any of the knee brace systems or assemblies disclosed herein can include one or more controllers. In some embodiments, dynamic bracing systems can include integrated electrical stimulation that can be configured for assisting in achieving joint flexion and/or extension. In some embodiments, one or more linear springs, torsion springs, and/or cam-based systems can be used to provide dynamic bracing options. In some embodiments, the controllers can be integrated and/or coupled with stays, joints, pivots or wraps of the knee brace system.

In some embodiments, this platform can comprise at least one stimulation system, one or more sensor systems, at least one display system and a coupled controller. Further, in some embodiments, the knee brace system can be controlled by and/or transfer data through the controller in a wired or wireless fashion. For example, in some embodiments of the invention, control electronics can include a pivotal joint configured to enable a brace of the knee brace system to flex (e.g., during the patient's flexion and extension). The pivotal joint can include a solenoid and an accelerometer to lock the brace (e.g., after sensing a stress). In one embodiment, the pivotal joint can include a digital positional encoder to determine an absolute position of the joint. In some embodiments, the positional encoder can enable adjustment of the physical resistance applied to the joint when the patient moves the joint.

In some embodiments of the invention, the outcomes of the therapy can be assessed by measuring joint space narrowing. In some further embodiments, the outcomes of the therapy can be assessed by measuring joint pain.

In some embodiments, the outcomes of the therapy can be assessed by measuring joint temperature, by means of one or more temperature sensors, multi-function IMUs, or other conventional sensors.

In some embodiments, the outcomes of the therapy can be assessed by measuring joint functional outcomes, including by monitoring a timed up and go test (TUG), and/or a six-minute walk test.

In some further embodiments, the outcomes of the therapy can be assessed by analyzing PROMs and/or measuring joint functional outcomes including, but not limited to, WOMAC osteoarthritis index, visual analog scale (VAS), knee injury and osteoarthritis outcome score (KOOS), KOOS JR, veterans rand 12 item (“VR-12”), and activities of daily living (ADL) scale.

In some embodiments, the outcomes of the therapy can be assessed by measuring muscle contraction forces using one or more conventional force sensors or gauges. In some embodiments, the force sensors or gauges can be coupled to a knee brace (or other suitable) therapy system.

In some embodiments of the invention, the outcomes of the therapy can be assessed by measuring muscle EMG using EMG electrodes or wearable textile sensors of at least one embodiments of a knee brace (or other suitable) therapy system disclosed herein.

Some embodiments include a device and method that provides NMES therapy to slow the progression of knee OA disease by making the quadriceps muscles stronger using an embodiment of a knee brace therapy system disclosed herein.

Some other embodiments include a device and method that provides NMES therapy to slow the progression of knee OA disease by creating an eccentric contraction of the muscle using at least one knee brace therapy system disclosed herein.

Some further embodiments include a device and method that provides NMES therapy and/or support devices to slow the progression of knee OA disease by reducing the impulsive loads and compressive forces on the knee joint during gait.

Some embodiments include a knee brace therapy system and method that provides NMES therapy to slow the progression of knee OA disease by reducing the incidence of joint degeneration, pain, and swelling.

Some embodiments include a knee brace therapy system and method that provides NMES therapy to slow the progression of knee OA disease by reducing the incidence for abnormal articular afferent information sent a motoneurons, and reducing the incidence for voluntary activation deficits of the muscles.

Some embodiments include a knee brace therapy system and method that provides NMES therapy to slow the progression of knee or other OA disease by providing real-time therapy results and outcomes to both patient and remotely to the patient's healthcare provider.

Some embodiments include a knee brace therapy system and method that provides NMES therapy to slow the progression of knee OA disease by predicting changes of the disease progression and joint health by measuring the muscle strength, and/or using patient database analytics and/or machine learning.

Some embodiments of the invention include a device that estimates a personalized dose of NMES therapy (intensity and duration) by measuring patient's muscle strength, disease stage, and/or by using a patient database analytics and/or machine learning.

Some embodiments of the invention include a device that estimates a patient's post-operative rehabilitation time and clinical outcomes based on pre-habilitation data (including NMES therapy intensity, duration, muscle strength, and ROM) by application of machine learning algorithms.

Some embodiments of the invention include a device that estimates a patient's risk level based on the pre-habilitation data (including NMES therapy intensity, duration, muscle strength, EMG, and ROM) by application of machine learning algorithms.

Some embodiments of the invention include a device that estimates correlations between or among patient demographics, NMES intensity, NMES duration, muscle strength, EMG, joint pain, joint flexion, joint extension, joint ROM by application of machine learning algorithms.

Some embodiments include a knee brace therapy system and method that can provide a unique LAMES waveform with specific pulse characteristics to provide strong contraction of the muscles while minimizing muscle fatigue and discomfort.

Some embodiments include a knee brace therapy system and method that can provide a unique pulse shape with specific pulse characteristics that generate forceful muscle contraction at a lower amplitude and longer duration than conventional therapy methods and devices.

Some embodiments include a knee brace therapy system and method that can provide a unique waveform that allows for slow and steady delivery of energy over a longer period of time to create a comfortable yet strong contraction without causing muscle fatigue.

Some embodiments include a knee brace therapy system and method that can provide a unique electrical stimulation waveform that allows for oscillation of contractions for different muscle groups to minimize muscle fatigue.

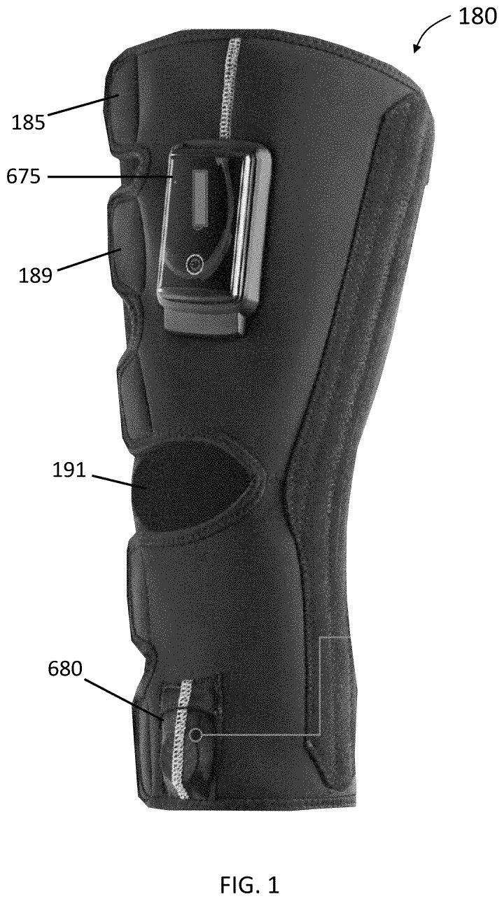

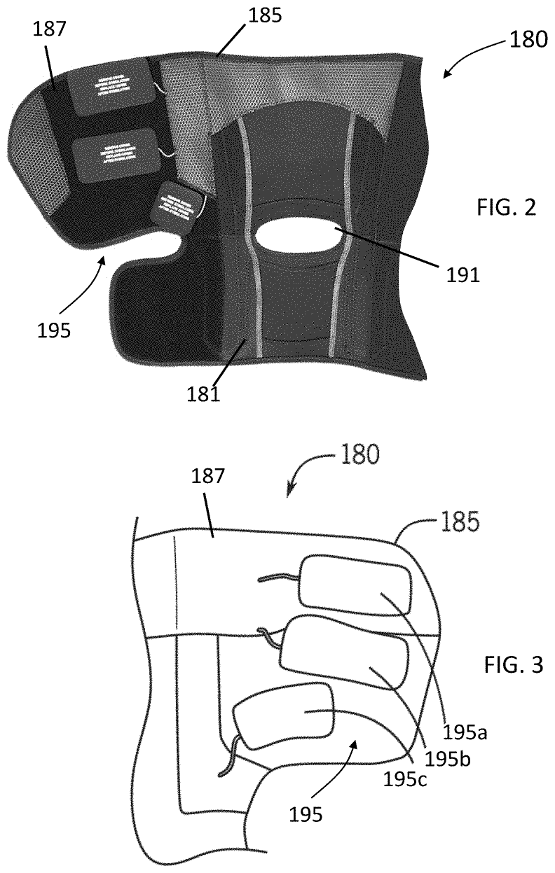

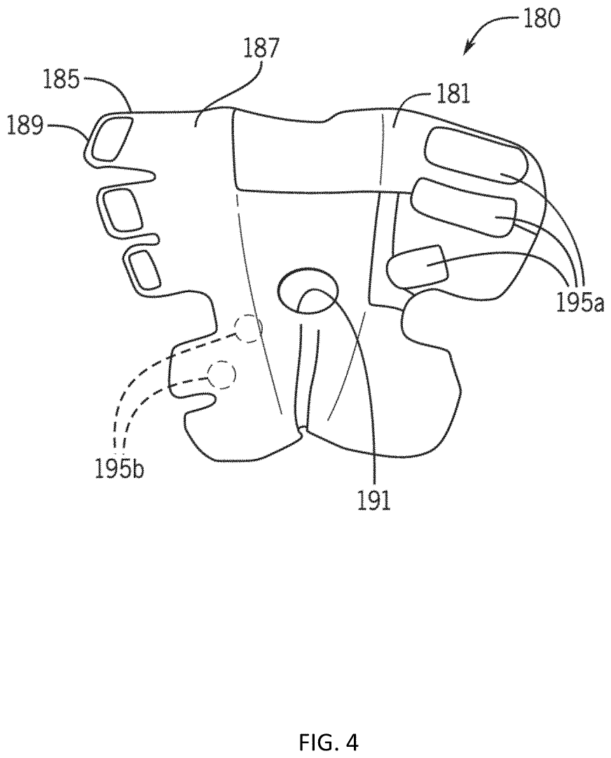

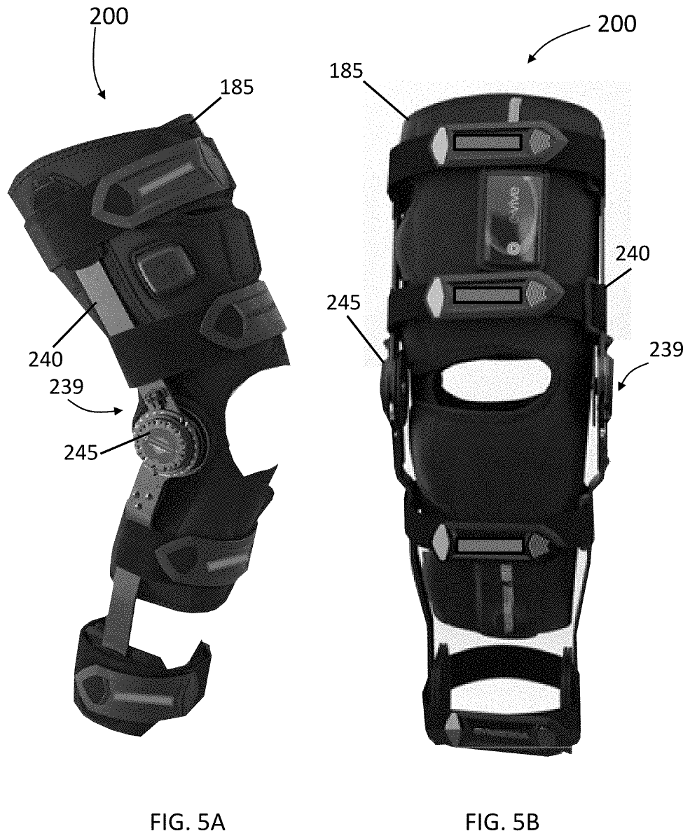

FIG. 1 illustrates a knee therapy system 180 in accordance with some embodiments of the invention, and FIGS. 2 and 3 illustrate partial views of an inner side of a knee wrap 185 of the knee therapy system 180 of FIG. 1 in accordance with some embodiments of the invention. Further, FIG. 4 illustrates a view of an inner side of a knee wrap 185 in accordance with some embodiments of the invention. Some embodiments of the knee therapy system 180 can include a knee brace that can include an attached, coupled, or integrated knee wrap 185, discussed below with respect to FIGS. 5A-5B, 6-7, and 9-10. Further details of the knee wrap 185 are shown in at least FIGS. 2-4. In some embodiments, the knee wrap 185 can comprises a flexible garment or wrap with one or more stays. In some embodiments, the knee wrap 185 can comprise a high-compression and non-slip material that is breathable. In some embodiments of the invention, the system 180 can comprise a knee wrap 185 that includes a non-slip compression material 187. In some embodiments, this material can assist in preventing movement of the knee wrap 180 when positioned on the wearer through friction and compression force. In some embodiments, the knee wrap 185 can include various extensions 189 to enable wrapping and attachment of the wrap 180 to the knee of the user, and can include various apertures to accommodate various portions of the wearer's body. For example, in some embodiments, the knee wrap 180 can include a popliteal cutout 191 to accommodate the structure and movement in the vicinity of the back of the wearer's knee.

Some embodiments of the invention include knee brace systems or assemblies that can capture data related to range of motion (also known as “ROM”). In some embodiments, range of motion data can be used prior to surgery to determine when the patient has recovered enough from an initial injury trauma to undergo surgery, potentially indicating that swelling and soft tissue mobility are at acceptable levels for surgery. In some further embodiments, range of motion data can be used after surgery to determine when the patient has recovered (and therefore can be used to determine the rate of recovery from surgery).

Some embodiments of the invention include knee brace systems or assemblies that can capture data related to knee gait. In some embodiments, gait related data can be used to estimate knee joint misalignment such as Varus and Valgus angles and gait speed.

In some embodiments, various electronics can be coupled to or integrated with the knee wrap 185. For example, some embodiments provide a knee therapy system 180 that can include at least one coupled sensor coupled to the inner or outer surface of the wrap 185 and/or to one or more stays with the ability to include three-axis movement. In some embodiments, one or more sensors can be integrated or coupled to at least a portion of the knee therapy system 180 and used to measure or monitor user parameters, track the functional characteristics of the knee brace system, and/or monitor the environment of the user, and/or measure absolute or relative position and/or movement of any portion of the knee brace system while attached to the user. Depending on the user's movement, the sensors can each move independently of each other in three dimensions.

As shown in the non-limiting embodiments of FIGS. 2-4, some embodiments include one or more sensors integrated into a wearable wrap or garment portion of a knee or other body part therapy system. For example, in some embodiments, the knee therapy system 180 can include a wrap 185 that can be used without a knee brace and can fully support the sensors and other components disclosed herein as being coupled to a knee brace. In some other embodiments, one or more sensors can be added to any rigid or flexible portion of the knee brace system.

In some embodiments of the invention, the knee wrap 185 can include one or more stimulation electrode or electrode pairs 195 such as quadriceps electrodes 195a and/or calf electrodes 195b. Moreover, in some embodiments, the electrode or electrode pairs 195 can be positioned on the inner surface 181 of the wrap 180 to enable contact with the skin of a wearer.

As used herein, in some embodiments, each stimulating electrode pair can comprise a first electrode structure having a first polarity, and a second electrode structure having a second polarity. The first and second polarities can be different so that the first and second electrode structures function to form an electrode pair capable of electrical stimulation. In some embodiments, the structure of the first electrode can be substantially the same or similar to the second electrode. In some other embodiments, the structures of the first and second electrodes can be different. In some embodiments, the electrodes are not limited to conventional electrode structures. For example, in some embodiments, one or more electrodes can comprise conductive material capable of transmitting signals efficiently or, in some embodiments, with significant loss or degradation while still providing sufficient signal strength for the particular application. As used herein, the terms “stimulating electrode” and “stimulating electrode pair” can be used interchangeably.

For example, in some embodiments, any of the aforementioned sensors can measure the position and/or movement and acceleration of any one of the sets of geometry of the knee therapy system 180 in any x, y, and/or z-axis. In some embodiments of the invention, the sensors can include an accelerometer such as one or more small solid-state or micro-electromechanical systems (MEMS) accelerometers, gyroscopes, and/or magnetometers that can be coupled to one or more portions of the knee brace system. In some embodiments, these sensors can measure/sense position and orientation, acceleration, velocity, vibration or shock along a single, or multiple axes. For example, some embodiments include an integrated 3-axis gyroscope, 3-axis geomagnetic sensor, and 3-axis accelerometer that can measure an absolute orientation vector in form of Quaternion or Euler angles.

In some embodiments of the invention, the sensors can comprise at least one Hall effect sensor. In some embodiments, the knee therapy system 180 can include one or more magnets coupled to portions of the knee therapy system 180 that can be used in combination with any conventional magnetic sensor. For example, some embodiments of the invention can comprise at least one Hall effect sensor can be used with one or more magnets to determine motion of at least a portion of the knee brace system. As just one example, in some embodiments, the sensor can determine rotation relative to a fixed point on a hinge of the knee therapy system 180 (e.g., when coupled to a stay as a portion of the knee therapy system 200 of FIGS. 5A-5B).

In some embodiments, any of the sensors and/or electrodes disclosed herein can be used to give active feedback to the patient about current range of motion. In some embodiments, range of motion data can be used to continually or periodically provide feedback to a user to encourage them to stretch muscles or move a joint during a recovery phase.

In some embodiments, tactile feedback can be provided whenever a user has exceeded a specified maximum range of motion. Further, in some embodiments, the knee brace system can be used to warn a user when they are hitting a range of motion that is not considered to be safe based on the user's stage of recovery. In some other embodiments, the knee brace system can incorporate dynamic resistance, spring rate, and/or force or damping if high accelerations or ranges of motion are detected in order to protect the joint. In some embodiments, this can be achieved using magneto-rheological fluids, inertia valve designs, piezoelectric materials, springs, shock absorbers, etc.

Some embodiments of the invention include kinematic data collection sensors for measuring the position and movement of a knee brace system. Further, in some embodiments, the knee brace system can include range of motion sensors for any knee brace system that includes one or more hinge features. In some embodiments, the sensors can include indexing points so that absolute position can be determined. Some embodiments of the invention can include proximity or contact based sensors to determine where set points on a hinge are in proximity of the sensor. In some embodiments, the sensor can be an optical (shadow, self-imaging, or interferometric) sensor, a magnetic sensor, an inductive sensor, a capacitive sensor, an eddy current sensor, a resistive sensor, a magneto-resistive sensor, an inductive sensor, an infrared sensor, an accelerometer sensor, an inclinometer sensor, a piezoelectric sensor, etc.

In some embodiments, joint range of motion can be measured by a knee therapy system using at least one optical camera coupled to the knee therapy system. In this instance, any movement of any portion of the knee therapy system can be tracked optically. Other methods can include the use of electroactive polymers and/or stretch sensitive fabrics coupled to a portion of a knee brace or wrap of the knee therapy system, and configured to track movement of any portion of the knee therapy system.

In some embodiments, joint range of motion can be measured by a knee therapy system using IMU, and/or at least one accelerometer, and/or at least one inclinometer, and/or at least one goniometers, and/or fiber optics.

In some embodiments, joint angles and rotation can be measured by a knee therapy system using at least one optical camera, and/or an IMU, and/or at least one accelerometer, and/or at least one inclinometer, and/or at least one goniometer, and/or at least one stretch-sensitive fabrics, and/or at least one fiber optics.

In some embodiments, joint shock impact can be measured using at least one accelerometer, and/or a piezoelectric sensor or piezo films (e.g., such as a PZT sensor), and/or electret films, and/or force sensitive resistors, and/or electroactive polymers, and/or pressure sensitive films, and/or a strain gauge.

In some embodiments, joint temperature can be measured using at least one thermocouple, at least one thermistor, at least one IR camera, at least one strain gauge, and/or a piezoelectric sensor or piezo films (e.g., such as a PZT sensor).

In some embodiments, muscle contraction force can be measured using at least one accelerometer, a piezoelectric sensor or piezo films (e.g., such as a PZT sensor), and/or electret films, and/or force sensitive resistors, and/or pressure sensitive films, and/or capacitive sensing, time/frequency analysis, and/or piezoelectric sensor or piezo films (e.g., such as a PZT sensor).

In some embodiments, the system can track body joints of a user including body joint movements by combining computer vision and machine learning technology. In some embodiments, the system can primarily focus on lower body joints (e.g., hip, knee and ankle), and can create a 2D plane where it can measure movement, time and angle of user's predefined activity. In some embodiments, the system can use a machine learning model which is trained to recognize a user's pose by drawing a skeleton (annotation) on a live camera feed from a user's mobile device (phone, tablet or other), and can provide instantaneous or prompt test feedback.

In some embodiments, the system can perform one or more of the following steps to compute the result for a given test including:

(i). take a live feed from camera as an RGB or other suitable image.

(ii). feed an image to a CNN (convolutional neural network).

(iii). a single pose decoding algorithm is used to decode or estimate a pose, pose confidence scores, body part and joint positions, and pose confidence scores from the model outputs.

(iv). calculate results for the tests (joint extension, flexion, ROM, GAIT or others) using model outputs or instructs users to redo a test if key-point confidence scores are not within the acceptable range.

(v). display results to a user on successful completion and upload results to the cloud or other database for further analysis.

In some embodiments, an EMG associated with each contraction can be measured using an EMG circuit, and/or an electrometer, and/or capacitive coupling, and/or inductive coupling. Some embodiments include a biofeedback system for simultaneous detection of EMG-triggered EMS by means of EMG electrodes, movement, or contraction detection using wearable wireless EMG sensors, pressure sensors, PZT sensors, force-sensitive sensor, strain gauge, etc.

Any of the above disclosed sensors or sensor combinations can be coupled to an external surface of any portion of the knee wrap or knee brace (e.g., such as the knee wrap 185 and or knee therapy system 200), including for example, to locations within the wrap and/or stays (e.g., on surfaces facing or coupled to the intended wearer and/or on surfaces facing away from the intended wearer). In some embodiments, sensors can be integrated with the knee brace by integrating into an internal portion of the knee brace or by coupling to an external surface of the knee brace. For example, in some embodiments, at least one stay can be coupled to an upper portion of a wrap (e.g., such as knee wrap 185) for positioning against, proximate or adjacent to the thigh of a user, and another stay can be coupled to a lower portion of a wrap for positioning against, proximate or adjacent to the lower leg of a user. In some embodiments, the knee brace can comprise a stay movable coupled to another stay about a pivot region. In some embodiments of the invention, the knee therapy system and/or any of the knee brace systems or assemblies disclosed herein can include systems and methods for determining positional data of any component or portion of the knee brace system.

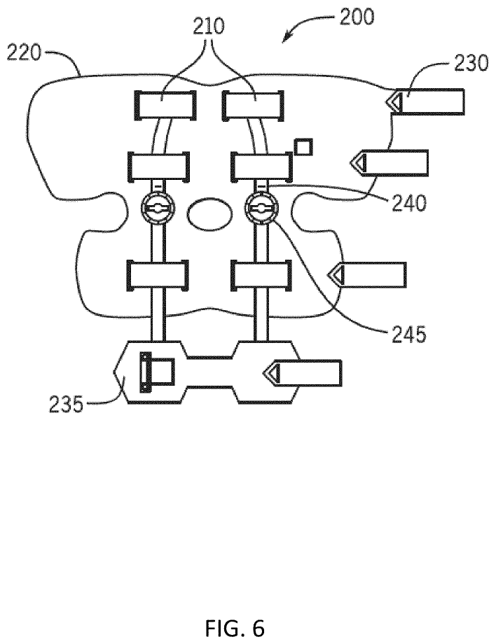

In some embodiments, one or more knee brace assemblies 239 can be integrated and/or coupled to a knee wrap 185 to form a combined modular orthopedic knee brace and conductive wrap. FIG. 5A illustrates a front side perspective view of a brace system 200 comprising a combined modular orthopedic brace 239 and conductive wrap 185 in accordance with some embodiments of the invention. FIG. 5B illustrates a front view of a brace system 200 comprising a combined modular orthopedic brace 239 and conductive wrap 185 in accordance with some embodiments of the invention. FIG. 6 illustrates a combined modular orthopedic brace 200 and conductive wrap assembly 220 in an open view position in accordance with some embodiments of the invention.

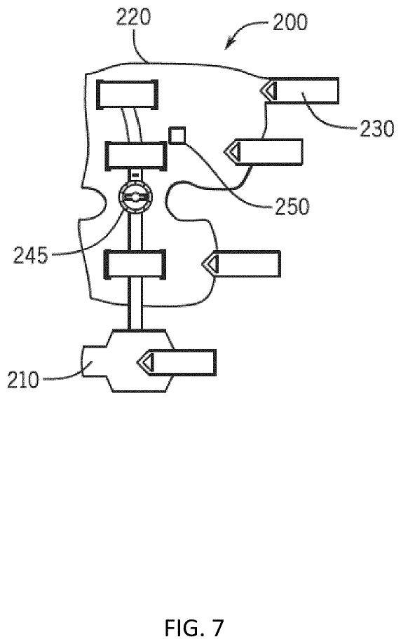

In some embodiments of the invention, for positioning, compression, and comfort, the wrap assembly 220 can include brace straps 230, malleolus pads 235, and a slide lock 240. Further, in some embodiments, a stimulation module can be coupled to the assembly 220 to enable application of stimulation therapy. For example, FIG. 7 illustrates an inner region of a brace system 200 showing two contact points used to determine if the brace 239 is being worn by a human and stimulation module 250 in accordance with some embodiments of the invention. Further, in some embodiments, the assembly can include a dial hinge 245 with ROM stops to enable customized fitting and therapy.

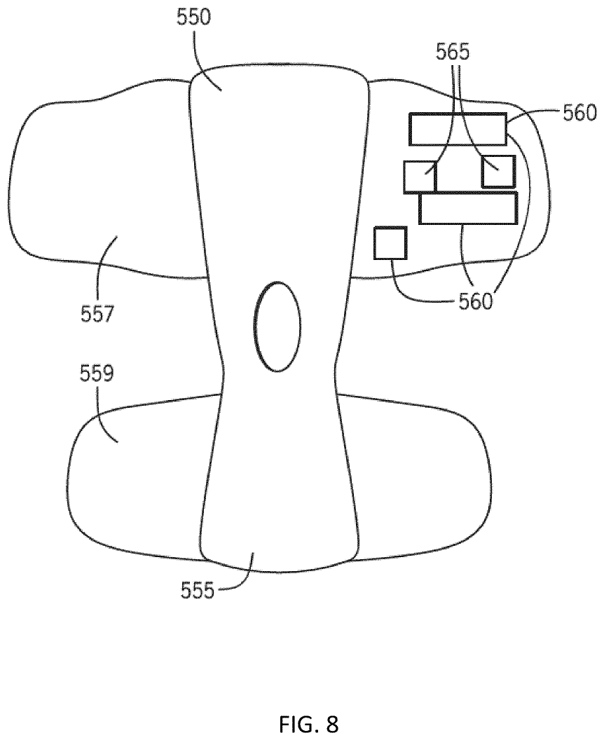

In some further embodiments, one or more sensors and/or electrodes can be coupled to various inner regions of the knee brace system. For example, FIG. 8 illustrates an inner region of a brace system 550 that can comprise a main body portion 555 and upper and lower strap portions 557, 559. In some embodiments, the brace system 550 can include electrodes on the inside of one of the strap portions 557, 559 that can be used to stimulate muscle groups. For example, in some embodiments, strap portion 557 can include a plurality of electrodes 560 positioned on various regions of the strap portion 557. Further, in some embodiments, either or both strap portions 557, 559 can include at least one contact sensor. For example, in some embodiments, the strap portion 557 can include at least one integrated or coupled contact sensor 565. In some embodiments, portions of the sensors 565 can comprise contact points that are located and configured at the outer surface of the inner region of the brace system 550. In some embodiments, the sensors 565 can comprise human contact sensors that can be used to determine if the brace is being worn by a human. In some embodiments, measurements from the sensors 565 can be used to provide patient compliance data where usage of the brace system is monitored and logged. In some other embodiments, the sensors can be used to monitor if the brace system is correctly positioned on the user. In some further embodiments of the invention, the measurement of position, movement, and/or acceleration of a portion of knee therapy system can be used to track the position and movement of the user. For example, in some embodiments, the system can be used to monitor a user to determine how much time the user spends in an upright position and/or in a supine position. In some embodiments, acceleration data from the brace system can be computed on a per limb basis which can be tallied as a running average. Further, in some embodiments, this average acceleration value can be used to directly correlate to the amount the patient is moving the limb, and can be used as key to identify a decrease in range of motion. For example, the lower the number, the lower the general level of movement of the user in total. In some embodiments, if the maximum flexion numbers received from the sensors are high, and the average acceleration value is very low, the user is sitting in place flexing a limb. However, if the average acceleration value number is very high, and the maximum flexion numbers are low, the user is moving around, but they are keeping the braced limb in a locked or nearly locked position with no or little movement at the joint.

In some embodiments, using any of the integrated or coupled sensors or accelerometers disclosed herein, free fall incidents can be determined by the one or more sensors of a knee brace therapy system and reported to computer system (e.g., such as a coupled computer or server or backend system or mobile device as disclosed herein). In some embodiments, the knee brace system can record the free falls to denote any time the brace (and the user) have fallen. Further, in some embodiments, the knee brace system can determine the height of the fall based on the duration and the rate of acceleration. In some embodiments, the knee brace system can determine if the user began to fall and subsequently caught themselves. Moreover, in some embodiments, the backend system can create and/or calendar a follow up requirement for a medical professional to determine if the fall did any damage.

In some further embodiments of the invention, patient compliance data obtained from the accumulated measurements from the sensors can be stored on a database (e.g., in a back-end computer system) and can be used by, for example, physicians or medical professionals to retrieve, review, and/or analyze the data from the knee brace system. In some embodiments, the physicians may utilize the data from the knee brace in the physician's analyses or recommendations to the patient. Further, in some embodiments, physicians may utilize the data from the knee brace system of one patient in recommendations to other patients with similar conditions or injuries. For example, if the physician tells a patient recovering from an ACL reconstructive surgery to execute one program for the first week, and to execute a second program for the second week, and if the physician sees significant improvements in the patient's strength in the patient's knee due to these programs, the physician will likely tell another patient recovering from a similar surgery to execute the same programs during the same time periods. In some embodiments, the physician can have the programs for the second patient updated remotely via a wired or wireless connection to the Internet or a private network. The physician can then obtain data from both patients to see how they are responding to the knee brace system and the programs being executed by the knee brace system.

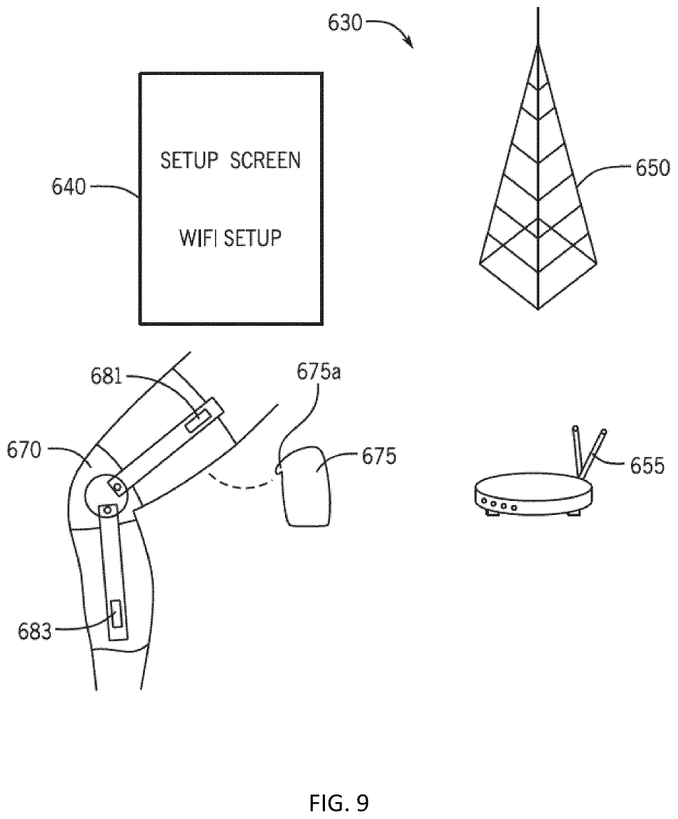

In some embodiments, the brace system can comprise control electronics that can include a communication module (e.g., transmitter or transceiver or wire) for communicating with one or more computing devices. For example, in some embodiments of the invention, any of the knee brace systems or assemblies described herein can be configured to transmit and/or receive information wirelessly. For example, FIG. 9 depicts a knee brace system wireless data transfer data architecture in accordance with some embodiments of the invention. FIG. 9 shows a representation of a wireless brace system 630 configurable for wireless collection of data from a knee brace assembly 670 including data communicated through a cellular 650 and/or a WiFi network 655 to a coupled or integrated controller 675 comprising a wireless antenna 675a. In some embodiments, one or more portions of the knee brace assembly 670 can include one or more sensors 681 (e.g., an accelerometer or other sensor as discussed earlier) coupled to stay 682 and/or sensor 683 coupled to stay 684 that can be coupled to the controller 675 to enable wireless transmission of data from and/or to the controller 675 and/or sensors 681, 683. Other embodiments include coupled or integrated sensor or sensors 680 as shown in at least FIG. 1. In some embodiments, the sensor or sensors 680 can be coupled to the controller 675 to enable wireless transmission of data.

In some embodiments, a graphical user interface (GUI) 640 can be used to control and/or monitor the function of various functional aspects of the wireless brace system 630, including any of the components in the system 630. In some embodiments, the controller 675 can comprise a rechargeable or battery powered power and control unit configured for stimulation and collection of sensor data.

In some embodiments, the controller 675 can manage sensing and/or stimulation of a patient wearing a brace system or garment (e.g., such as wireless brace system 630). In some embodiments of the invention, the controller 675 can be configured to (a) apply at least one stimulation sense pulse to the patient's tissue using at least one sensor and/or electrode, (b) measure at least one electrical parameter from the patient's tissue related to power dissipation of the sense pulse in the tissue, (c) adjustably apply the at least one stimulation pulse to the patient's tissue based at least in part on the measured power dissipation. In some embodiments, the at least one stimulation pulse can be adjustably controlled by the at least one controller to maintain a constant power output to the patient's tissue based at least in part on the at least one electrical parameter. In some embodiments, the steps (a) through (c) can be repeated at least once.

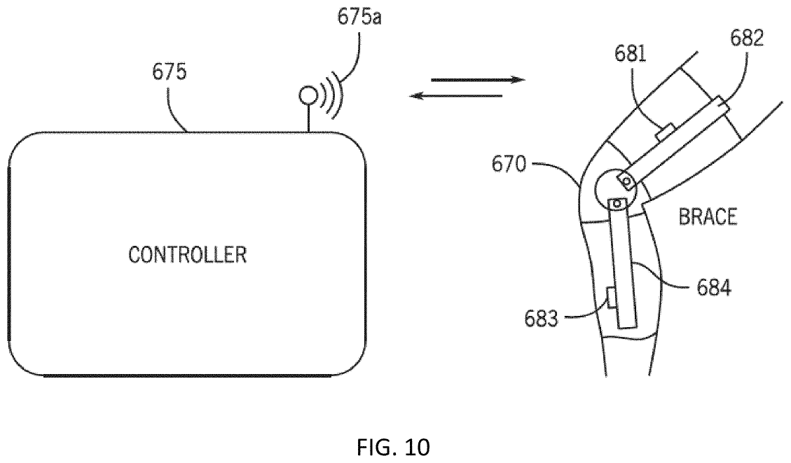

As one non-limiting example embodiment, FIG. 10 depicts wireless data transfer data between the knee brace assembly 670 and the controller 675 in accordance with some embodiments of the invention. In some embodiments, a wireless RF transmission from the knee brace assembly 670 can be of sufficient power to enable reliable operation and transmission of data from the brace system with adequate bandwidth while minimizing tissue propagation characteristics and specific absorption rate (to avoid tissue heating) and reduce exposure of the user to near-field and far-field RF transmission. In some embodiments, the knee brace assembly 670 can be configured to transmit and/or receive an RF transmission including, but not limited to, a zero generation wireless signal, a first generation wireless signal, a second generation wireless signal, a third generation wireless signal, a fourth generation wireless signal, a fifth generation wireless signal, any global positioning satellite signal (such as “GPS” or “GLONASS”), an industrial, scientific, and medical (ISM) frequency bands (e.g., 2400-2493.5 MHz), a Bluetooth® wireless signal (such as IEEE 802.15.4 Bluetooth® class II), RFID electromagnetic radiation, a WiFi wireless signal, a two-way radio RF signal, a UHF or VHF signal (such as a citizen's band radio signal or other radio signal emitted from a ‘walkie-talkie’ type device), high-speed and millimeter wave signals, and a near-field wireless signal. Bluetooth® is a computing and telecommunications industry specification that details how mobile devices can easily interconnect with each other and with non-mobile devices using a short-range wireless connection, and the name Bluetooth® is a registered trademark of Bluetooth SIG, Inc.

In some embodiments, the controller 675 can comprise a computer system or device. In some embodiments, the knee brace assembly 670 can be configured to communicate (e.g., wirelessly or via a wired connection) with a computing device that may perform the function of the controller 675. Examples of the computing device include, but are not limited to, personal computers, digital assistants, personal digital assistants, mobile phones, wearable technology devices (e.g. smart watches, activity monitors, heart rate monitors, glasses, cameras, etc.), smartphones, tablets, or laptop computers. In some embodiments, the computing device can be the patient's device or a device associated with a medical professional. Both types of devices can enable the medical professional to retrieve and analyze data transmitted from the brace system. In one embodiment, this data is transmitted in real-time, so that the medical professional can analyze the data and/or adjust the brace at any time. For example, in some embodiments, the patient can access data using a mobile application on his device. In some further embodiments, a physician and/or therapist can access data via a web portal. In some embodiments, any data accessed through from any of the brace systems described herein, including any data collected or channel through a controller such as controller 675 can be secured using one or more conventional encryption methodologies. In some embodiments, the protocols and method for data transfer as described are HIPAA compliant.

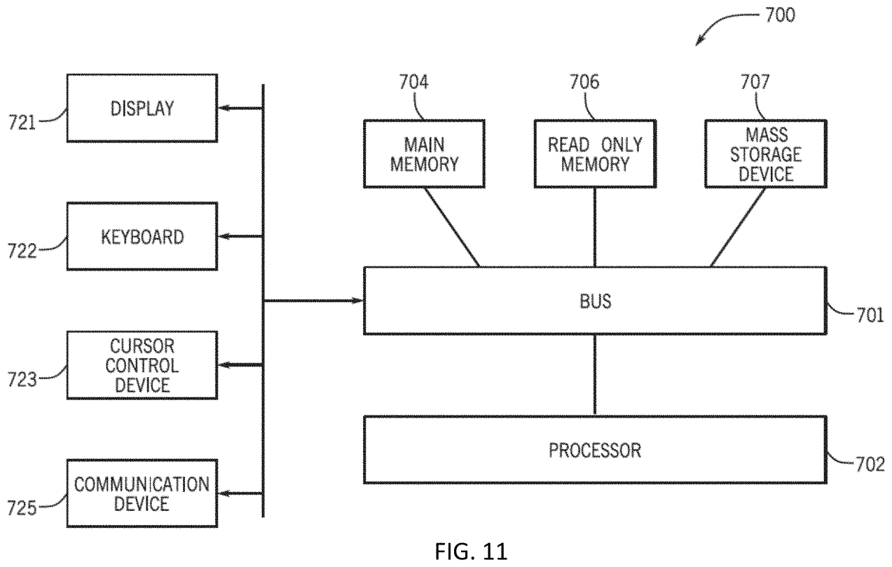

Some embodiments include a brace system that can also comprise brace control electronics that can be configured to provide the NMES via a program selected from a plurality of programs. In at least one embodiment of the invention, the brace control electronics can be configured to receive, via a receiver, a selection of the program (e.g., from the patient, from a medical professional, etc.) In one embodiment, the medical professional can prevent patient control of the brace (e.g., for a period of time). Referring to FIG. 11, in some embodiments, any of the brace systems or assemblies described herein can electronically couple with a computer system 700 that can be configured to transfer data from and/or to the brace system. In some embodiments, a brace system (such as brace system 670) can communicate with the computer system 700 using a controller, such as controller 675. In some embodiments, the controller 675 can function as an internet transceiver coordinating and routing data between the brace and the computer system 700. In some embodiments, the system 700 comprises the controller 675. In some embodiments of the invention, the computer system 700 can be a local computer system (e.g., a computer system within the user's home) that can be configured to receive and/or send information to the brace system 670. In some embodiments, the computer system 700 can include a bus 701 for communicating information between the components in the computer system 700. Further, in some embodiments, at least one processor 702 can be coupled with the bus 701 for executing software code, or instructions, and processing information. In some embodiments of the invention, the computer system 700 further compromises a main memory 704, which can be implemented using random access memory (RAM) and/or other random memory storage devices. In some embodiments, the main memory 704 can be coupled to the bus 701 for storing information and instructions to be executed by the processor 702. Further, in some embodiments, the main memory 704 also can be used for storing temporary variables, NMES program parameters, or other intermediate information during the execution of instructions by the processor 702. In some embodiments, the computer system 700 can also include a read only memory (ROM) and/or other static storage device coupled to the bus 701 for storing static information and instructions for the processor 702. In some embodiments of the invention, the computer system 700 can include one or more peripheral components enabling user interaction with the system 700. For example, in some embodiments, the system 700 can include a cursor control device 723, such as a conventional mouse, touch mouse, trackball, track pad, or other type of cursor direction keys for communicating direction information and command selection to the processor 702 and for controlling movement of a cursor on the display 721. Further, the system 700 can also include at least one keyboard 722 for data input, and facilitation of command and control of the various aspects of the system 700, and at least one communication device 725 operatively coupled to the processor 702 via the bus 701.

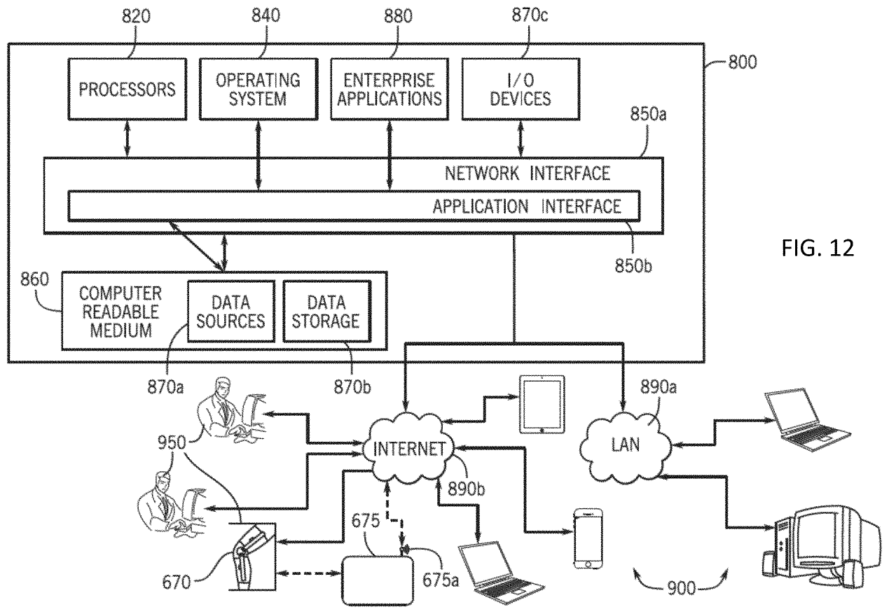

In some embodiments, any of the brace systems or assemblies described herein (including the brace system 670) can be coupled to and transfer data from and/or to a computer system that is configured to receive and/or send information to the brace system and any coupled computer system. Turning to FIG. 12, in some embodiments, a computer system 800 can comprise a backend system that can be used as a host computer for storing information measured and sent by the brace system. In some embodiments of the invention, the information can be received and/or sent between the brace system and the computer system 800 using the computer system 700 (i.e., a local computer system and/or controller that can be configured to receive and/or send information to the brace system locally). In some further embodiments, the information can be received and/or sent between the brace system and the computer system 800 directly (e.g., using a cellular wireless transmission). Further, in some embodiments, the brace can communicate with the computer system 800 and the computer system 700 using a controller, such as controller 100. In some embodiments, the controller can function as an internet transceiver coordinating and routing data between the brace and the computer systems 700, 800.

In some embodiments of the invention, the system 800 can include at least one computing device, including at least one or more processors 820. In some embodiments, some processors 820 can include processors 820 residing in one or more conventional server platforms. In some embodiments, the system 800 can include a network interface 850a and an application interface 850b coupled to at least one processors 820 capable of running at least one operating system 840. Further, the system 800 can include the network interface 850a and the application interface 850b coupled to at least one processor 820 capable of processing one or more of the software modules 880 (e.g., one or more enterprise applications). In some embodiments, the software modules 880 can comprise a server-based software platform. In some embodiments, the system 800 can also include at least one computer readable medium 860. In some embodiments, at least one computer readable medium 860 can be coupled to at least one data storage device 870b, and/or at least one data source 870a, and/or at least one input/output device 870c.

In some embodiments, the invention can also be embodied as computer readable code on a computer readable medium 860. In some embodiments, the computer readable medium 860 can be any data storage device that can store data, which can thereafter be read by a computer system. Examples of the computer readable medium 860 can include hard drives, network attached storage, read-only memory, random-access memory, FLASH based memory, CD-ROMs, CD-Rs, CD-RWs, DVDs, magnetic tapes, other optical and non-optical data storage devices, or any other physical or material medium which can be used to tangibly store the desired information or data or instructions and which can be accessed by a computer or processor.

In some embodiments, the computer readable medium 860 can also be distributed over a conventional computer network. For example, in some embodiments, the computer readable medium 860 can also be distributed over and/or accessed via the network interface 850a. In this instance, computer readable code can be stored and executed in a distributed fashion using the computer system 800. For example, in some embodiments, one or more components of the system 800 can be tethered to send and/or receive data through a local area network (“LAN”) 890a. In some further embodiments, one or more components of the system 800 can be tethered to send or receive data through an Internet 890b (e.g., such as a wireless or wired internet). In some embodiments, at least one software module 880 running on at least one processor 820 can be configured to be coupled for communication over a network 890a, 890b.

In some embodiments, one or more components of the network 890a, 890b can include one or more resources for data storage and retrieval. This can include any computer readable media in addition to the computer readable medium 860, and can be used for facilitating the communication of information from one electronic device to another electronic device. Also, in some embodiments, the network 890a, 890b can include wide area networks (“WAN”), direct connections (e.g., through a universal serial bus port), other forms of computer-readable medium 860, or any combination thereof. In some embodiments, the software modules 880 can be configured to send and receive data from a database (e.g., from a computer readable medium 860 including data sources 870a and data storage 870b that can comprise a database). Further, in some embodiments, data can be accessed and received by the software modules 880 from at least one other source.

In some embodiments of the invention, one or more components of the network 890a, 890b can include a number of user-coupled devices 900 such personal computers including for example desktop computers, laptop computers, digital assistants, personal digital assistants, cellular phones, mobile phones, smart phones, wearable technology devices (e.g. smart watches, activity monitors, heart rate monitors), glasses, cameras, pagers, digital tablets, internet appliances, and other processor-based devices. In general, a client device can be any type of external or internal devices such as a mouse, a CD-ROM, DVD, a keyboard, a display, or other input or output devices 870c. In some embodiments, at least one of the software modules 880 can be configured within the system 800 to output data to a user via at least one digital display. Further, in some embodiments, various other forms of computer-readable medium 860 can transmit or carry instructions to a user interface such as a coupled device 900, including a router, private or public network, or other transmission device or channel, both wired and wireless.

In some embodiments, the system 800 as described can enable one or more users 950 to receive, analyze, input, modify, create and send data to and from the system 800, including to and from one or more software modules 880 running on the system 800. Some embodiments include at least one user 950 accessing one or more modules, including at least one software module 880 via a stationary I/O device 870c through a LAN 890a. In some other embodiments, the system 800 can enable at least one user 950 accessing software module 880 via a stationary or mobile I/O device 870c through an internet 890a.

In some embodiments, the brace system or controller (e.g., any of the knee brace systems described earlier) can comprise software modules that are upgradeable. In some embodiments, the software modules can be upgraded by an Internet download (for example through the Internet 890b shown in FIG. 12). In some embodiments of the invention, the Internet download can comprise accessing at least one or more software modules stored in a cloud-based storage location. In some embodiments, the brace system can access a cloud-based storage location to perform periodic software updates and/or to store brace system data, and/or data from a brace system controller, and/or user data (i.e., data from a brace system attached to the user).

With the above embodiments in mind, it should be understood that some embodiments of the invention can employ various computer-implemented operations involving data stored in computer systems (such as the system 800 shown in FIG. 12). In addition, in some embodiments, the above-described applications of the monitoring system can be stored on computer-readable storage media (such as computer readable medium 860). These operations are those requiring physical manipulation of physical quantities. Usually, though not necessarily, these quantities take the form of electrical, electromagnetic, or magnetic signals, optical or magneto-optical form capable of being stored, transferred, combined, compared and otherwise manipulated.

Any of the operations described herein that form part of the invention are useful machine operations. The invention also relates to a device or an apparatus for performing these operations. The embodiments of the invention can be defined as a machine that transforms data from one state to another state. The data can represent an article, that can be represented as an electronic signal and electronically manipulate data. The transformed data can, in some cases, be visually rendered onto a display, representing the physical object that results from the transformation of data. The transformed data can be saved to storage generally or in particular formats that enable the construction or depiction of a physical and tangible object. In some embodiments, the manipulation can be performed by one or more processors 820. In such an example, the processors 820 can transform the data from one thing to another. Still further, the methods can be processed by one or more machines or processors that can be connected over a network. Each machine can transform data from one state or thing to another, and can also process data, save data to storage, transmit data over a network, display the result, or communicate the result to another machine. Further, the brace system as described will result in a large quantity of data that must be manipulated, transformed, refined, reduced, or changed from one state to another to be able to efficiently resolve into meaningful segments of data that the user or clinician can utilize and make medical based judgments upon. In one embodiment, the brace system or controller includes software that performs a data collection and pre-filtering algorithm that stores data onto storage media only after some of the desired conditions have been met (e.g. the user is wearing the brace and movement is occurring above/below a desired threshold, or ROM data is captured only when user is vertical, or in periodic time points throughout the day such as once per minute or during user awake hours, etc.) In another embodiment, the computer system 800 performs the data reduction and pre-filtering function. Computer-readable storage media (such as computer readable medium 860) as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

In some embodiments of the invention, the initiation of wireless data transfer from and/or to the brace system (e.g., by using cellular transfer of data) can be autonomous and/or semi-autonomous and can be configured to not require user configuration. For example, in some embodiments, the device can automatically check in when powered on. In some embodiments of the invention, the brace system can include a backend system comprising one or more servers that are looking for devices to check in at times for set usage. The backend system is the system of record for the patient compliance data. In some embodiments, if the device does not check in, the backend system or controller can send a message the patient (or anyone else on a contact list) to indicate that device should be checked in.

Some embodiments of the invention can include uploading data to the backend by coupling to a smart device or a computer. By way of example, in some embodiments, Bluetooth® products can be used to provide links between any of the brace systems or assemblies described herein and mobile computers, mobile phones, portable handheld devices, wearable technology devices (e.g. smart watches, activity monitors, heart rate monitors, glasses, cameras, etc.), personal digital assistants (PDAs), tablets, and other mobile devices and connectivity to the Internet. In some embodiments, wireless transmission can occur via a Bluetooth® wireless signal from the brace system to the smart device or computer. In some embodiments, a user interface screen can be used to enable pairing of devices by using the Bluetooth® protocol. In some further embodiments, uploading data to the backend can occur by coupling to WiFi® to connect to the user's home network or office network. In some embodiments, this will require the creation of a user interface screen that allows the user to select a wireless network to connect to and to provide credentials to connect to that network.

In some embodiments of the invention, the brace system can utilize wireless protection schemes to control data access to and from the brace system. This can protect patient confidentiality and to protect the security of the data. Some embodiments include protection against unauthorized wireless access to device data and control. In some embodiments, this can include software and/or hardware enabled protocols that maintain the security of the communications while avoiding known shortcomings of existing older protocols (including for example the Wired Equivalent Privacy (WEP)). In some embodiments, usage data that is transmitted from the devices (via Bluetooth®, WiFi, or via other means) can be encrypted to ensure that only the patient or the patient's physician can obtain access to this medical information. The encryption can be done via either software executing on the processor or via external hardware that processes the data before it is transmitted. In one embodiment, each set of logs is uniquely tied to the device that created them. This can be done by the device tagging the data being transmitted from the device with a unique identifier associated with the device. The unique identifier is set either by the processor or by an external component of the system (e.g., a UUID chip).

In some embodiments, the wireless collection can include wireless collection of compliance data. For example, in some embodiments, brace system data comprising a user's compliance to certain daily movements and/or one or more physiotherapy or exercise routines can be wirelessly monitored and recorded. In some embodiments, the brace system can comprise a wireless collection of compliance data and can include creation of a record of all instances that brace system sensor determines a patient is wearing the brace system. In some embodiments, this can include stored data (e.g., data that has previously been measured and stored in a volatile or non-volatile memory by the brace system). For example, this can include a wireless collection of kinematic data, including data such as orientation data and acceleration data. In some embodiments, the brace system can continue to store and transmit data when the user is not wearing the brace system. In some embodiments, the data can be ignored, and in other embodiments, the data can be stored and/or wirelessly transmitted. In some embodiments, the brace system can wirelessly transmit data from the brace system to at least one telemedicine system. In some embodiments, the brace system can wirelessly transmit data from the brace system to at least one physiotherapist and/or physiotherapist system.

In some embodiments, one or more brace control programs can be selected by a medical professional or patient that can be dynamic (e.g., changeable or variable, not a fixed frequency, not fixed timing, not a fixed waveform, etc.) and can cause different types of EMS to be executed on different parts of the patient's body. For example, if the feedback data obtained and rendered by the brace system from the brace system's control electronics indicates that the patient's vastus medialis oblique muscles are getting stronger while the patient's distal central hamstring (or, in another embodiment, the patient's calf muscle) is not getting stronger, a medical professional (e.g., physician or physical therapist) can instruct, via one or more of these programs, the brace system to execute a predetermined brace control program. In some embodiments, the brace system can include specific programs for the first week after surgery, specific programs for the first month after surgery, specific programs for arthritis, etc.

In some embodiments, feedback can be collected on the back side of the feedback loop, after it has passed through the user. Some embodiments include control systems that are configured to maintain a constant output from the system. In some embodiments, the system can be configured to maintain a constant output as is passes through the user. In some embodiments, during the course of NMES, the conductive properties of a user's tissues change. In some embodiments of the invention, the brace system can comprise a feedback loop that compensates for tissue changes by attempting to keep the output constant. As the resistance rises, the system can induce more current to keep the power dissipation levels constant in the system. In some embodiments, if the resistance gets beyond a certain point the voltage of the system will spike to attempt to break through the high resistance element and allow current to flow.

Some embodiments of the invention can include systems configured for obtaining biological feedback. In some embodiments, biological feedback can be provided by one or more biological feedback sensors coupled to a user using a brace system. In some embodiments, one or more of the brace systems or assemblies described herein can comprise at least one biological feedback sensor configured to provide biological feedback data from a user. For example, in some embodiments, the human contact sensors shown in FIG. 8 can comprise one or more biological feedback sensors positioned within the inner region of a brace. In some embodiments, these sensors can be proximity or contact sensors capable of determining if a device (e.g., such as a brace) is being worn by a user. Further, for example, electrical sensors can be included to determine the impedance between sensors to determine if the device is attached to human skin. In some further embodiments, other sensors can be used such as blood pressure sensors, blood oxygen level sensors, heart rate sensors, laser or ultrasound based sensors for measuring movement of tissues or fluids, hydration sensors that measure the interstitial fluid levels to determine hydration levels, force or pressure sensors for measuring the muscle activity/response, or electromyography type sensors to measure muscle recruitment from the electrical stimulation therapy, or to measure the level of muscle fatigue. In some further embodiments, by measuring the hydration levels of the user, the system can tune the electrical stimulation signals to be more optimized or less painful for the user or provide feedback to the user to drink more fluids.

In some further embodiments, the biological feedback sensor can comprise one or more temperature sensors. In some embodiments, one or more temperature sensors can be coupled to or integrated with a brace system, and used to monitor temperature proximate the user. In some embodiments, one or more temperature sensors can be used in combination with NMES therapy and used to sense temperatures proximate stimulation electrodes. In some embodiments of the invention, one or more temperature sensors can be used in combination with NMES therapy and used for feedback control. For example, in some embodiments, the brace system can include a closed loop feedback system that provides electrical muscle stimulation (EMS) to a joint of a human patient in response to feedback from a sensed temperature. In some embodiments, the brace system can include one or more sensors in physical contact with the skin of the patient and configured to obtain a sense and/or obtain information from a region of the skin and/or of a NMES electrode contacting the skin of a patient.

For example, in some embodiments, one or more temperature sensors can be used to sense temperature proximate one or more NMES electrodes. In some embodiments, the brace system can also include brace control electronics in communication with the sensor(s) to form a closed loop system via a combination of bracing the joint and electrical muscle stimulation (EMS). Further, in some embodiments, the brace control electronics can be configured to receive temperature measurements of the skin of the patient and/or of one or more of the electrodes, and is further configured to apply a current/voltage/power onto the skin based on the temperature. For example, NMES can be reduced or increased based at least in part a temperature measurement from the one or more temperature electrodes. In some embodiments, using one or more temperature sensors to sense temperature proximate one or more NMES electrodes, where the sensed temperature is used for control of NMES, NMES burns can be substantially reduced or eliminated. In some further embodiments, one or more temperature sensors sensing changes in a user's body and/or body core temperature can be used to estimate a user's activity level, or the presence of an infection or other condition.

Some embodiments of the invention include knee brace assemblies with systems for monitoring for the presence or concentration of at least one chemical, biochemical marker or other analyte. In some embodiments, analytes can include naturally occurring or synthetic compounds or molecules, and/or metabolites. For example, in some embodiments, the brace system can include a blood oxygen sensor apparatus configured for measuring the oxygen content of blood. In some embodiments, a brace system configured with blood oxygen monitors can enable an assessment of blood pooling and can be used to prevention of deep vein thrombosis (DVT), and other potentially fatal events such as pulmonary embolism, extremity edema, and so on.

In some further embodiments, one or more of the knee brace systems or assemblies described herein can include a sensor apparatus configured for measuring nicotine, nicotine metabolites, and/or other drugs or drug metabolites including stimulants, depressants, hallucinogens, designer drugs, and anabolic steroids. In some embodiments, at least one of the brace systems or assemblies described herein can comprise one or more sensors configured to detect one or more of these substances in-vivo and to notify the healthcare professional since they may affect the healing and rehabilitation process. In some other embodiments, the brace system can be configured with sensors to detect the immediate environment of a user. For example, in some embodiments, nicotine from first-hand or second-hand smoke can be sensed using one or more brace system chemical sensors and used to determine if the user may have smoked and/or has been exposed to elevated levels of tobacco smoke.

In some embodiments, any of the knee brace systems or assemblies described herein can include at least one sensor configured to measure a heart-rate of a user. For example, in some embodiments, at least one heart rate sensor can be used to determine if patients are performing prescribed exercises and/or physical therapy. Further, in some embodiments, at least one heart rate sensor can be used to determine a user's overall activity level (used for healing and data correlation). In some further embodiments, lung and/or breath sensors can be used to provide data for a VO₂ max calculation, and provide additional data on activity level. In some embodiments, the brace system can include at least one heart-rate sensor integrated with a portion of a brace. In other embodiments, the brace system can include at least one heart-rate sensor coupled to and adjacent to or some distance from the brace.

In some further embodiments, one or more of the knee brace systems or assemblies described herein can include a non-invasive blood pressure sensor configured to measure arterial blood pressure continuously or intermittently. In some further embodiments, a user's heart-rate can be measured in addition to sensing the user's blood pressure. In some embodiments, one or more of the brace systems or assemblies described herein can include at least one blood pressure sensor integrated with a portion of a brace. In other embodiments, the brace system can include at least one blood pressure sensor coupled to and adjacent to or some distance from the brace.

In some further embodiments of the invention, some one or more of the knee brace systems or assemblies described herein can comprise an electromyography sensor, a strain gage sensor or other sensor configured to measure strains continuously or intermittently. In some embodiments, these measurements can be used to assess motion, deflection, or provide quantifiable data of muscle growth, muscle contraction, or forces, torques or pressures resulting from a muscle contraction. The muscle contraction may be voluntary or involuntarily elicited via electrical muscle stimulation. In some embodiments, the data collected from the electromyography sensor or strain gage sensor can be utilized in a closed loop feedback control methodology in order to optimize/customize the electrical stimulation parameters to provide the most efficient or strongest muscle contraction for that patient. The data can also be utilized by the healthcare provider to fine tune the treatment programs based on the patient's data captured from the electromyography or strain gage sensor.

Any of the above disclosed sensors or sensor combinations, brace systems, wraps or assemblies described herein can be used for pelvic floor muscle therapy. Some embodiments include a digital health based non-invasive surface electrical stimulation therapy and a biofeedback system with data collection and sharing capabilities. Some embodiments include a surface EMS therapy to strengthen weakened pelvic floor muscles by external stimulation of pudendal nerve branches in the pelvic area.

Some further embodiments include an integrated biofeedback system that provides an interactive real-time monitoring and visualization of the muscle contractions. For example, some embodiments include a mobile application and database that provides a tool for managing the EMS delivery, biofeedback monitoring and display, and collection, storage of data. Some embodiments can utilize the EMS with closed-loop feedback control and power dissipation characteristics, with wireless external EMS to stimulate the pelvic floor by activation of nerve branches of pudendal nerve in thigh and glutes area in upper legs and hip region.

Some embodiments include an application of external surface electrodes, a conductive garment or a wearable garment or wireless electrodes in form of a wrap or shorts. Some embodiments of the invention include a garment configured to be worn by the user and comprising a mounting module having an array of connection regions, and a set of electrodes or biometric sensors coupled to the garment and configured to communicate with the array of connection regions to send EMS and receive biometric signals indicative of muscle activity of the user.

Some further embodiments include a biofeedback system for simultaneous detection of biofeedback signals of pelvic floor activation by means of EMG, movement, or contraction detection using wearable wireless EMG sensors, pressure sensors, PZT sensors, force-sensitive sensor, strain gauge, etc.

Some applications that include an integrated biofeedback system that provides an interactive real-time monitoring and visualization of the muscle contractions can include a portable control module configured to couple to the garment for delivery of wireless EMS, control of EMS channels and intensities, collection, storage, and display of detected biofeedback signals (EMG, pressure, and movement).

Some embodiments also include a display of a patient's compliance with EMS therapy, a user's leakage tracking management in association with EMS therapy. Some further embodiments include collection of a user's reported outcomes such as a quality of life score. Some embodiments include Kegel exercises or other pelvic floor muscle strengthening exercises. In some embodiments, the system can track, review, and share data with providers. In some embodiments, the data may be analyzed in real time and feedback may be provided to the user based on the analysis. In some embodiments, the analysis may be used to alter behavior of the user and/or therapy. Some embodiments include a delivery of personalized therapy dose (intensity and duration) based on the collected health data and application of machine learning algorithms).

It will be appreciated by those skilled in the art that while the invention has been described above regarding particular embodiments and examples, the invention is not necessarily so limited, and that numerous other embodiments, examples, uses, modifications and departures from the embodiments, examples and uses are intended to be encompassed by the description, figures, and claims therein.

1-22. (canceled)

23. A system for stimulating a pudendal nerve comprising:
a flexible garment,
a plurality of electrodes,
an electrical circuit, and
at least one controller;
wherein the flexible garment is in the form of wearable shorts;
wherein the shorts are configured and arranged to cover at least a portion of a user's thigh and glutes;
wherein the plurality of electrodes are coupled to or integrated with the flexible garment;
wherein each of the plurality of electrodes are external surface electrodes configured and arranged to contact the surface of a patient's skin;
wherein at least one of the plurality of electrodes is positioned adjacent a pudendal nerve branch when the flexible garment is worn by the user;
wherein the position of the at least one of the plurality of electrodes is configured to activate the pudendal nerve branch; and
wherein the system is configured and arranged to stimulate a pelvic floor through the activation of the pudendal nerve branch.

24. The system of claim 23,
wherein the plurality of electrodes includes at least one active electrode and at least one receiving electrode forming the electrical circuit with control electronics of the at least one controller.

25. The system of claim 24,
wherein the electrical circuit is configured and arranged to measure an electrical parameter using the at least one active electrode and at least one receiving electrode.

26. The system of claim 25,
wherein a stimulation current or voltage applied onto the pudendal nerve branch between the at least one active electrode and at least one receiving electrode is based on at least one program and at least one electrical parameter measured through the at least one active electrode and at least one receiving electrode.

27. The system of claim 23,
wherein the at least one controller is configured and arranged to
(a) apply a sense electrical pulse to the tissue,
(b) measure the at least one electrical parameter from the tissue,
(c) using at least one of the active electrodes, adjustably apply a stimulation pulse to the pudendal nerve branch based at least in part on the measured electrical parameter.

28. The system of claim 27,
wherein the system is a non-invasive surface electrical stimulation therapy.

29. The system of claim 27,
further comprising a strain gage sensor configured to provide strain measurements to the at least one controller.

30. The system of claim 29,
wherein the system is configured to use the strain measurements to provide muscle contraction data.

31. The system of claim 29,
wherein the system is configured to use the strain measurements to provide muscle growth data.

32. A system for stimulating a pudendal nerve comprising:
a garment,
at least one active electrode, and
at least one receiving electrode;
wherein the garment is configured and arranged to cover at least a portion of a user's thigh and glutes;
wherein the at least one active electrode and at least one receiving electrode are coupled to the garment;
wherein the at least one active electrode and at least one receiving electrode are external surface electrodes configured and arranged to contact the surface of a patient's skin;
wherein at least one of the at least one active electrode and at least one receiving electrode are positioned adjacent a pudendal nerve branch when the garment is worn by the user;
wherein the position of at least one of the at least one active electrode and at least one receiving electrode is configured to activate the pudendal nerve branch; and
wherein the system is configured and arranged to stimulate a pelvic floor through the activation of the pudendal nerve branch.

33. The system of claim 32,
further comprising:
an electrical circuit, and
at least one controller;
wherein the at least one active electrode and at least one receiving electrode are configured and arranged to form the electrical circuit with control electronics of the at least one controller; and
wherein the electrical circuit is configured and arranged to measure at least one electrical parameter using the at least one active electrode and at least one receiving electrode.

34. The system of claim 33,
wherein the measured at least one electrical parameter is used by the controller to determine a stimulation current to activate the pudendal nerve branch.

35. The system of claim 33,
wherein the at least one controller is configured and arranged to
(a) apply a sense electrical pulse to the tissue,
(b) measure the at least one electrical parameter from the tissue,
(c) using at least one of the active electrodes, adjustably apply a stimulation pulse to the pudendal nerve branch based at least in part on the measured electrical parameter.

36. The system of claim 32,
wherein the system is a non-invasive surface electrical stimulation therapy.

37. The system of claim 33,
further comprising a strain gage sensor configured to provide strain measurements to the at least one controller.

38. The system of claim 37,
wherein the system is configured to use the strain measurements to provide muscle contraction data.

39. The system of claim 37,
wherein the system is configured to use the strain measurements to provide muscle growth data.
